SEQUENCE LISTING (18 sequences)

SEQ ID NO: 1 - DNA, 34 bp, Artificial Sequence
  Primer
ggaagatctatgaaatcatacaaacttaataacc

SEQ ID NO: 2 - DNA, 31 bp, Artificial Sequence
  Primer
tcccccgggggcttcaatgctaagatgagag

SEQ ID NO: 3 - DNA, 31 bp, Artificial Sequence
  Primer
aactatcttgtctacaagaaaagtgggacta

SEQ ID NO: 4 - DNA, 31 bp, Artificial Sequence
  Primer
tagtcccacttttcttgtagacaagatagtt

SEQ ID NO: 5 - DNA, 19 bp, Artificial Sequence
  Primer
acatggaaggctgagaacc

SEQ ID NO: 6 - DNA, 20 bp, Artificial Sequence
  Primer
ccaagaaataatccactacg

SEQ ID NO: 7 - DNA, 21 bp, Artificial Sequence
  Primer
cctctcatcttagcattgaag

SEQ ID NO: 8 - DNA, 22 bp, Artificial Sequence
  Primer
ccaagaaataatccactacgat

SEQ ID NO: 9 - DNA, 20 bp, Artificial Sequence
  Primer
agcgagagagaaagcgtttc

SEQ ID NO: 10 - DNA, 20 bp, Artificial Sequence
  Primer
ccttccatgtctcagattcc

SEQ ID NO: 11 - DNA, 20 bp, Artificial Sequence
  Primer
gaagagcaccctgttcttct

SEQ ID NO: 12 - DNA, 20 bp, Artificial Sequence
  Primer
tggcgtacaaggagagaaca

SEQ ID NO: 13 - DNA, 22 bp, Artificial Sequence
  Primer
tggttcacgtagtgggccatcg

SEQ ID NO: 14 - DNA, 21 bp, Artificial Sequence
  Primer
ccgtcccgcaagttaaatatg

SEQ ID NO: 15 - DNA, 24 bp, Artificial Sequence
  Primer
gcctttttcaaactcaagttgcac

SEQ ID NO: 16 - DNA, 24 bp, Artificial Sequence
  Primer
gagtttattatgtgtgtgcgtgga

SEQ ID NO: 17 - DNA, 20 bp, Artificial Sequence
  Primer
actcatctccacgcacacac

SEQ ID NO: 18 - DNA, 20 bp, Artificial Sequence
  Primer
aatctcgaaccgggaatgat

(19) United States
(12) Patent Application Publication
(10) Pub. No.: US 2012/0005774 A1
(43) Pub. Date: Jan. 5, 2012

(54) USE OF TREHALASE TO OBTAIN DROUGHT RESISTANCE IN PLANTS

(75) Inventors: Patrick van Dijck, Zichem (BE); Lies Vandesteene, Welle (BE); Nelson Avonce, Haasrode (BE)

(73) Assignees: KATHOLIEKE UNIVERSITEIT LEUVEN, K.U. LEUVEN R & D, Leuven (BE); VIB VZW, Gent (BE)

(21) Appl. No.: 13/148,655

(22) PCT Filed: Feb. 8, 2010
(86) PCT No.: PCT/EP10/51500

§ 371 Date: Sep. 12, 2011

Related U.S. Application Data
(60) Provisional application No. 61/207,188, filed on Feb. 9, 2009.

(51) Int. Cl. A01H 1/06

(52) U.S. Cl. 800/278

(57) ABSTRACT

The present invention relates to the use of trehalase to obtain drought and/or salt resistance in plants. More specifically, it relates to genetically modified constructs, transformed into plants and resulting in overexpression of trehalase, whereby the transformed plants show a significantly better drought resistance during the drought period, and a better recovery when water is supplied after the drought period.

### CROSS-REFERENCE TO RELATED APPLICATIONS

This is a national phase entry under 35 U.S.C. §371 of International Patent Application PCT/EP2010/051500, filed Feb. 8, 2010, published in English as International Patent Publication WO 2010/089392 A1 on Aug. 12, 2010, which claims the benefit under Article 8 of the Patent Cooperation Treaty to U.S. Provisional Patent Application Ser. No. 61/207,188, filed Feb. 9, 2009.

### TECHNICAL FIELD

The present invention relates to the use of trehalase to obtain drought and/or salt resistance in plants. More specifically, it relates to genetically modified constructs, transformed into plants and resulting in overexpression of trehalase, whereby the transformed plants show a significantly better drought resistance during the drought period, and a better recovery when water is supplied after the drought period.

### BACKGROUND

The disaccharide trehalose (α-D-glucopyranosyl-1,1-α-D-glucopyranoside) is a non-reducing sugar found in a variety of organisms including plants. The role of trehalose has often been associated with stress resistance, particularly resistance to desiccation. Jang et al. (2003) describe the construction of transgenic rice plants with increased abiotic stress tolerance by the expression of a fusion of the Escherichia coli genes for trehalose-6-phosphate synthase and trehalose-6phosphate phosphatase. Suarez et al. (2008) obtained improvement of drought tolerance in common beans by overexpression of trehalose-6-phosphate synthase. In those studies, the accumulation of trehalose is promoted by increasing the biosynthesis.

However, apart from the biosynthetic pathway, the level of trehalose may be influenced by trehalose degradation. Trehalase is the only known enzyme in Arabidopsis able to degrade trehalose to two glucose units. The expression of its gene, AtTRE1, is significantly up-regulated during specific plant development stages, such as flowering and senescence, and during stress conditions. Because of the intimate connection between trehalose metabolism and plant vital processes, stringent level control of trehalose, or indirectly of T6P, by trehalase activity might be crucial in the fine regulation of these metabolites/signals and, subsequently, in the control of plant growth and development. Trehalase may also mobilize stress-induced trehalose levels to allow subsequent cellular repair, as is the case in lower organisms. Moreover, high trehalase activity may also prevent perturbations of carbon metabolism by external trehalose, released by pathogenic or symbiotic microbes. However, despite the involvement in multiple processes, little is known about the exact role of trehalase in higher plants.

EP0784095 discloses trehalose accumulation in potato upon inhibition of trehalase, either by addition of the trehalase inhibitor Validamycin A, or by expressing trehalase anti-sense RNA. However, these inhibition experiments were carried out in trehalose-6-phosphate synthase overexpressing plants, making it difficult to make a distinction between the effect of the overexpression of the synthase and that of the inhibition of the trehalase. WO2008095919 discloses the use of overexpression of a trehalase gene to confer nematode resistance to plants. However, no reference to drought or salt tolerance was made. On the contrary, it was indicated that higher trehalose concentration results in increased tolerance to drought and stress.

### DISCLOSURE

Surprisingly, we found that overexpression of trehalase resulted in a better tolerance of drought, and a better growth upon rewatering after a period of drought. Knock-out of the trehalase gene caused a decrease in drought tolerance.

A first aspect of the invention is the use of a recombinant trehalase gene to modulate drought and/or salt tolerance in plants. “Modulation drought” and/or “salt tolerance,” as used herein, can be an increase or a decrease in drought and/or salt tolerance. “Trehalase,” as used herein, means a glycoside hydrolase, converting trehalose into glucose. Trehalases are known to the person skilled in the art and include, but are not limited to, bacterial, fungal and plant trehalases. Preferably, trehalase is an Arabidopsis trehalase; even more preferably, it is the Arabidopsis trehalase TRE1 (genbank accession number NM_{—}118536). A “recombinant trehalase gene,” as used herein, means that the nucleic acid encoding trehalase has been isolated, was optionally modified, and introduced into a plant, resulting in modified drought and/or stress tolerance. Recombinant trehalase genes comprise, but are not limited to, knock-out genes, genes with modified, preferably optimized, coding sequence, genes with a modified promoter, antisense genes, and genes encoding RNAi. “Optimized coding sequences,” as used herein, include, as a non-limiting example, codon optimation for better translation, as well as mutations that may increase the specific activity of the protein. Due to the modification, the fragment introduced in the plant may be only a fragment of the trehalase gene. However, modification of the gene is optional. As a non-limiting example of the use of a trehalase gene without modification, the gene of one species, even a non-plant species, may be introduced in the plant. The introduction can be a single copy, or several copies can be introduced. Alternatively, the plant may be transformed with the endogenous gene to obtain a higher copy number of the gene, and so a higher expression of the trehalase protein. Preferably, that use is the overexpression of the trehalase gene, resulting in an improved drought and/or salt tolerance. Preferably, overexpression is resulting in improved drought tolerance. “Drought tolerance,” as used herein, comprises drought tolerance during cultivation, as well as drought tolerance after harvesting, resulting in a better shelf-life of the harvested plant. As a non-limiting example, drought tolerance after harvest may result in a better shelf-life of ornamental flowers, or in a better shelf-life of vegetables, preferably green vegetables such as, but not limited to, lettuce and spinach.

Preferably, overexpression of trehalase is specifically in the stomatal guard cells. “Specific expression in the stomatal guard cells,” as used herein, means that the expression is largely biased toward the stomatal guard cells; preferably, there is expression in the stomatal guard cells and no expression in the mesophyllic or epidermal cells of the plant leafs or plant stem, but there may be expression in parts of the root or the flowers. Such specific overexpression can be realized by the endogenous Arabidopsis thaliana TRE1 promoter.

Another aspect of the invention is the use of trehalase overexpression to obtain drought and/or salt tolerance in plants. Methods to obtain trehalase overexpression are known to the person skilled in the art and include, but are not limited to, increasing the copy number of trehalase genes, placing the trehalase coding sequence under control of a strong promoter, or removing repressing elements from the promoter. Trehalase overexpression may be obtained by mutagenesis and selection of an endogenous trehalase in a plant. Preferably, trehalase overexpression is obtained by isolating a trehalase gene, adapting it for overexpression and transforming it into a plant. Preferably, overexpression is resulting in improved drought tolerance. Preferably, trehalase is placed after a stomata-specific promoter, and overexpression is specifically in the stomatal guard cells.

Still another aspect of the invention is the use of trehalase overexpression to obtain improved growth upon rewatering after a drought period. “Rewatering,” as used herein, means that after a period of drought, the plant is exposed again to water. “Improved growth,” as used herein, includes, amongst others, a shorter adaptation period after the start of the rewatering, and better growth after adaptation.

Still another aspect of the invention is the use of a trehalase according to the invention, preferably the use of a recombinant trehalase gene according to the invention, to modulate the stomatal function. Stomatal function can be modulated by modulating the aperture of the stomatal cells, and/or the number of stomatal cells per leaf surface unit, and/or the stomatal index. Preferably, modulation of the stomatal function is realized by modulating the aperture of the stomata. Preferably, the use of trehalase is overexpression and modulation of the stomatal function is a limitation of the opening of the stomata. It is clear for the person skilled in the art that a limitation of the opening of the stomata, and/or a decrease in number of stomatal guard cells per leaf surface, and/or a decrease of stomatal index, can limit water loss and result in drought resistance, at least as long as a decrease of one factor is not compensated by an increase of another factor.

### BRIEF DESCRIPTION OF THE FIGURES

FIG. 1: Arabidopsis AtTRE1 mutant lines. Panel A, Cartoon of the insertion sites of GT tre KO and SA TRE OX lines in gDNA of Arabidopsis. The exact (full lines) and predicted (dotted lines) insertion sites of SA TRE OX are shown. For GT tre KO, prediction insertion site was confirmed. Positions of primer sets are also shown; black boxes are exons. Panel B, Relative trehalase expression of GT tre KO lines, compared to wild-type (wt) Arabidopsis thaliana ecotype Landsberg erecta (Ler). Panel C, Relative trehalase expression of SA TRE OX lines, compared to wt Arabidopsis thaliana ecotype Colombia (Col). For Panels B and C, real-time PCR results normalized to actin are shown from one representative line. Homozygous seedlings were grown for 10 days on 1× MS (1% suc, 12 hours-12 hours day/night cycle). Data are means±SE, n=2, with ±100 seedlings per experiment.

FIG. 2: Trehalase activity of AtTRE1 Arabidopsis mutant lines. Trehalase activities (nkat/g protein) were determined: Panel A, in adult leaves of wt Col and SA TRE OX plants, grown in soil; Panel B, in mixtures of young and ripe silique material of wt Ler and GT tre KO, grown in soil (data are mean values of mixed plant material from different plants per line).

FIG. 3: Growth phenotypes of AtTRE1 mutants on trehalose. Panel A, GT tre KO lines and wt Ler with Val A. Panel B, SA TRE OX lines and wt Col with Val A. Panel C, GT tre KO lines and wt Ler without Val A. Panel D, SA TRE OX lines and wt Col without Val A. Approximately 30 seedlings/treatment were grown on vertical plates, 1× MS without suc, in a 12 hours-12 hours day/night cycle, for 10 days with 0, 0.5, 1, 5, 10, or 25 mM trehalose. Data are means±SE.

FIG. 4: Growth phenotypes of AtTRE1 mutants on high trehalose. Seedlings grown in the same conditions as described in FIG. 3, without Val A, and with higher concentration of trehalose: 50, 100, 150, and 200 mM. Representative seedlings are shown.

FIG. 5: Carbohydrate use of AtTRE1 mutant seedlings. Panel A, SA TRE OX plants and wt Col plants grown on horizontally oriented plates with 1× MS, 0%, 3%, 6% sue or glc, 16 hours-8 hours, 10 days. Panel B, GT tre KO plants and wt Ler plants (same conditions as in Panel A). Panel C, SA TRE OX plants and wt Col plants grown on vertically oriented plates in continuous light with 1% sucrose, 0.5× MS, 6 days. Panel D, GT tre KO plants and wt Ler plants (same conditions as in Panel C). Representative seedlings are shown.

FIG. 6: Leaf growth of AtTRE1 mutants at different diurnal rhythm. Panel A, Leaves of wt Ler (first line) and GT tre KO plants (two different lines), 12 hours-12 hours day/night cycle. Panel B, Leaves of wt Ler (first line) and GT tre KO plants (two different lines), 16 hours-8 hours day/night cycle. Panel C, Leaves of wt Col (first line) and SA TRE OX plants (two different lines), 12 hours-12 hours day/night cycle. Panel D, Leaves of wt Col (first line) and SA TRE OX plants (two different lines), 16 hours-8 hours day/night cycle. Cotelydons, and rosette leaves are placed in order of appearance. Plants 4 weeks old, grown in soil. Representative seedlings are shown.

FIG. 7: Reproduction of AtTRE1 mutants. Panel A, Earlier flowering of GT tre KO plants compared to wt Ler plants, grown in soil, 4 weeks old (16 hours-8 hours). Panel B, View of plant development of wt Ler and GT tre KO plants and Panel C, wt Col and SA TRE OX plants, grown in soil for 7 weeks old (16 hours-8 hours). Panel D, Detail of flowers and siliques of wt Ler and GT tre KO plants. Phenotypes were observed several times, and representative plants are shown.

FIG. 8: Seedling development of AtTRE1 mutants at elevated temperature. Germination and growth of SA TRE OX and GT tre KO seedlings and, respectively, wild-types in 33° C. (continuous low light, 1× MS, 1% suc, 10 days). Representative seedlings are shown.

FIG. 9: Drought responses of AtTRE1 OX and KO Arabidopsis plants. Drought stress responses of plants, grown in soil, in normal watered conditions for 4 weeks, stopped watering for 2 weeks (Panels A, B) and a couple of days after rewatering (Panels C, D). Representative plants are shown.

FIG. 10: Localization of AtTRE1 in mesophyll protoplasts. Panel A, Light microscopy image of protoplast using dark interference contrast. Panel B, Localization of AtTRE1 with GFP-fluorescence, mainly in the plasma-membrane. Panel C, The auto-fluorescence of chloroplasts.

FIGS. 11A and 11B: AtTRE1 expression pattern in 10-day-old seedlings (grown on 1× MS), using promoter-GUS/GFP single insertion lines after GUS staining. FIG. 11A: expression in leaf A, leaf 2 and leaf primordia; FIG. 11B: expression in stomatal guard cells.

FIG. 12: Stomata closure and opening assay of AtTRE1 OX and KO lines. Differences in opening of stomata in trehalase overexpression lines (OX) compared with the trehalase knock-out (KO) and wild-type plants.

FIGS. 13A and 13B: Stomata and pavement cell counting assay of AtTRE1 OX and KO lines. Number of stomata (FIG. 13A) and pavement cells (FIG. 13B) counted on a sample of 0.142 mm² for trehalase overexpressing plants (OX) compared with the knock-out line (KO) and wild-type plants.

FIG. 14: Stomatal index of AtTRE1 OX and KO lines. The stomatal index corresponds to the ratio of stomata per total number of cells in the epidermis (# stomata/(2×# stomata+# pavement cells).

### DETAILED DESCRIPTION OF THE INVENTION

### EXAMPLES

Materials and Methods to the Examples

Arabidopsis Plants Used in this Study

TABLE I
plant lines

| Name | Ecotype | Source |
|---|---|---|
| SA TRE OX (SALK_151791) | Colombia | ABRC* |
| GT tre KO (GT_16843) | Landsberg erecta | CSHL* |

*From the ordered lines, homozygous plants were obtained in this study

Primers Used in this Study

TABLE II
Cloning and site-directed mutagenesis primers

| Primer | Sequence | RE | SEQ ID NO: |
|---|---|---|---|
| AtTRE1fw | GGAAGATCTATGAAATCATACAAACTTAATAACC | BglII | 1 |
| AtTRE1rv | TCCCCCGGGGGCTTCAATGCTAAGATGAGAG | SmaI | 2 |
| AtTRE1717sdmfw | AACTATCTTGTCTACAAGAAAAGTGGGACTA | | 3 |
| AtTRE1717sdmrv | TAGTCCCACTTTTCTTGTAGACAAGATAGTT | | 4 |

abbreviations: fw (forward), ry (reverse); RE (restriction enzyme).

TABLE III
Real-time PCR primers and semi-quantitative
RT-PCR primers

| Name primer | Sequence | SEQ ID NO: |
|---|---|---|
| AtTREP1fw | ACATGGAAGGCTGAGAACC | 5 |
| AtTREP1rv | CCAAGAAATAATCCACTACG | 6 |
| AtTREP2fw | CCTCTCATCTTAGCATTGAAG | 7 |
| AtTREP2rv | CCAAGAAATAATCCACTACGAT | 8 |
| AtTREP3fw | AGCGAGAGAGAAAGCGTTTC | 9 |
| AtTREP3rv | CCTTCCATGTCTCAGATTCC | 10 |
| ACTIN2fw | GAAGAGCACCCTGTTCTTCT | 11 |
| ACTIN2rv | TGGCGTACAAGGAGAGAACA | 12 |

TABLE IV
Primers to check insertion in Arabidopsis mutants

| | | | SEQ |
|---|---|---|---|
| | | | ID |
| Primer | Sequence | Mutant line | NO: |
| Lba1 | TGGTTCACGTAGTGGGCCATCG | SALK | 13 |
| Ds3 | CCGTCCCGCAAGTTAAATATG | GT | 14 |
| AtTRELP1 | GCCTTTTTCAAACTCAAGTTGCAC | SA TRE OX | 15 |
| AtTRERP1 | GAGTTTATTATGTGTGTGCGTGGA | SA TRE OX | 16 |
| AtTRELP2 | ACTCATCTCCACGCACACAC | GT tre KO | 17 |
| AtTRERP2 | AATCTCGAACCGGGAATGAT | GT tre KO | 18 |

Abbreviations: SA (SALK lines); GT (Genetrap line), LP (left primer), and RP (right primer).

Plasmids Used in this Study

Trehalase CDS was cloned (source material: siliques and flowers of wild-type Arabidopsis thaliana ecotype Colombia) in plant vector pGFP (Kovtun et al., 1998; 2000; Hwang and Sheen, 2001). pGFP=pUC18 modified for GFP fusion. CDS sequence was blasted against TAIR Arabidopsis Genome Initiative Coding Sequence datasets (www.arabidopsis.org) and mutations, leading to amino acid changes, were corrected by site-directed mutagenesis (sdm).

### Growth Media and Growth Conditions

### Vapor-Phase Sterilization of Arabidopsis Seeds

Transgenic and wild-type Arabidopsis, ecotype Col and ecotype Ler seeds were vapor-phase sterilized according to a protocol adapted from Ye et al. (1999). Seeds were placed in open micro-centrifuge tubes and put inside a desiccator jar, where bleach-chlorine fumes sterilize the seeds for a period of 3 hours-9 hours (bleach+concentrated HCl). Sterile water is added afterwards working in laminar flow, followed by a 48-hour cold treatment under constant light for imbibition and stratification.

### Growth in Soil

Vapor-sterilized seeds were sown, after a 48-hour stratification period, on wet soil and germinated for two days in high humidity. In general, they were grown in 12 hours-12 hours, 16 hours-8 hours day/night cycle or continuous light (21° C., 60% humidity, 100 μmol s⁻¹ m⁻²).

### In Vitro Growth

Vapor-sterilized seeds were sown after a 48-hour stratification period in vertically or horizontally oriented Petri dishes on a full strength (or mentioned otherwise) Murashige and Skoog Salt Mixture including vitamins (MS) medium (4.3 g/l) with MES (0.5 g/l) and solidified with Phytagar (8 g/l) (Duchefa, Haarlem, The Netherlands). The media was enriched with sucrose/glucose (0-6%) or trehalose, depending on the tested conditions, which are specified. Val A was added in a concentration of 10 μM.

### Selection of Plant Transformants and Homozygous Plant Lines

For homozygous plant selection, SALK lines and GT lines (with the NTPII marker gene as selection marker) were screened on 1×MS+Kanamycin (50 μg/ml, Sigma-Aldrich). For growth on MS media with selective media, seeds were germinated for seven days on MS and antibiotics, positives were transferred to soil to flower and to set seeds.

### Molecular, Physiological and Biochemical Techniques

RNA Extraction from Plants and cDNA Preparation

Plant samples (seedlings) were harvested and immediately frozen in liquid nitrogen. RNA extraction of plant material was performed with Trizol reagent (Invitrogen), according to the manufacturer's instructions. For each sample, reverse transcription (RT) was performed using 1 μg of total RNA (Reverse Transcription System, Promega).

Plasmid DNA Isolation from Bacterial Cells

Mini preparation—CTAB method (Del Sal et al., 1988)

Mini preparation—GenElute™ Plasmid Miniprep kit (Sigma-Aldrich)

Maxi preparation—Pure Yield Plasmid Maxiprep System (Promega)

Rapid Genomic DNA Isolation from Arabidopsis Plants

After grinding a medium size leaf with screwdriver-pestle in 100 μl TPS buffer (100 mM Tris-HCl, pH 9.5, 1 M KCl, 10 mM EDTA), the samples were incubated at 70° C. for 20-30 minutes. The debris was pelleted by centrifugation at top speed for 10 minutes at RT and supernatans transferred to a new Eppendorf tube. For further PCR reactions, 0.5 to 1 μl supernatans was used in a 20-50 μl reaction.

### Polymerase Chain Reactions (PCR)

### Standard PCR Reactions

PCR reactions were performed using different polymerase kits, according to manufacturer's recommendations; High-Fidelity Taq Polymerases (Roche), Phusion High-Fidelity DNA polymerase (FINNZYMES) and Ultra Pfu HF DNA polymerase (Stratagene) for cloning and ExTaq (TAKARA) for diagnostic PCR.

Purification of PCR Products

Equal amount of PCI was added to the PCR product (45 μl-45 μl), vortex, and centrifuged at maximum speed, for 5 minutes. To the transferred supernatans, NaAc 3M (3.6 μl) and ice-cold pure ethanol (109 μl) was added, vortex, and spinned down for 1 minute at maximum speed. After additional wash with ice-cold 80% ethanol, the pellet was dried out and dissolved in 12.5 μl H₂O. PCR products could also be purified with using the Wizard SV Gel and PCR Clean-UP System (Promega, USA), according to manufacturer's guidelines.

### Semi-Quantitative RT-PCR

PCR was performed on cDNA samples equally diluted to 250 ng/μl and a control gene was used as control. The PCR product was run on 1% agarose gel and visualized with SYBR Safe DNA gel stain (Invitrogen).

### Real-Time PCR

25 μl real-time PCR reaction was composed of 12.5 μl Platinum SYBR Green qPCR SuperMix-UDG (Invitrogen), 0.5 μl of ROX reference dye, 1.25 μl of each primer of 500 nM stock solution and 5 μl of cDNA mix (100 ng/μl). The PCR program comprised an initial denaturation of 2 minutes at 95° C., amplification by 50 cycles of 15 seconds at 95° C., 1 minute at 58° C. The expression levels were analyzed with ABI prism and were all normalized to ACTIN2 expression.

Site-Directed Mutagenesis (sdm)

Site-directed mutagenesis (sdm) was performed according to the Stratagene's QuickChange Site-Directed Mutagenesis kit. As such, point mutations, insertions and deletions were created in a plasmid with Ultra Pfu HF DNA polymerase (Stratagene).

### Transient Expression in Arabidopsis Mesophyll Protoplasts

Three to four week-old Arabidopsis plants (in soil, 12 hours-12 hours day/night cycle, under low light, 50-75 μmol m⁻² s⁻¹) with healthy, unstressed, and well-expanded true leaves were used. This technique is detailed described by Yoo et al. (2007) (genetics.mgh.harvard.edu/sheenweb/protocols_reg.html). Briefly, after the removal of cell walls, protoplasts are released, washed and collected. DNA-PEG4000 (platelets, Fluka)-Calcium transfection is performed using 2×10⁴ protoplasts per 10-20 μg of endotoxin-free (maxiprep) plasmid DNA. The transfected protoplasts are incubated for 6 hours in the light before harvesting.

Subcellular localization in mesophyll protoplast cells of the protein of interest can be determined by fluorescence microscopic analysis, using the GFP-tagged vectors.

### Trehalase Measurements in Plant Material

Crude leaf material (300 mg, mix of plant material/different plants) was homogenized and powdered on dry ice, suspended in 2 ml ice-cold extraction buffer (0.1 M MES/K⁺, pH 6; 1 mM PMSF 2 mM EDTA, 10 mg/gFW insoluble PVP, polyvinylpyrrolidone). The suspension was vortexed and centrifuged (13000 rpm, cold, 10 minutes). The supernatant was dialyzed (BRL microdialysis system) ON at 4° C. against MES buffer pH 7 with 50 μl CaCl₂. Trehalase activity was determined by a method described by Pernambuco et al. (1996) adapted to microtiter plates. 10 μl dialyzed sample was added to 50 μl trehalose buffer (250 mM trehalose, 25 mM MES, pH 7, 50 μl CaCl₂). The microtiter plate was incubated for 30 minutes at 30° C. and reaction was stopped by boiling 5 minutes at 95° C. warm waterbath. Glucose was determined using glucose oxidase-peroxidase method by adding 200 μl GOD-PAP (Dialab) (blancs: residual glucose content in each sample determined by boiling samples before trehalose addition). A505 was measured after 15 minutes of incubation at 30° C. The amount of protein was determined using Lowry method with BSA as standard (Lowry et al., 1951).

### Example 1

### Isolation of Arabidopsis AtTRE1 Mutants

The transposon insertion Genetrap (GT) line GT 16843 (A. thaliana, ecotype Ler), was originally produced in the Martienssen's Cold Spring Harbor Laboratory (Sundaresan et al., 1995; Martienssen, 1998). Based on the flanking sequences of the insertion site obtained by TAIL-PCR (Liu et al., 1995), line GT_{—}16843 was predicted to carry a unique insertion of a GT transposable Ds element, 395 by downstream of the ATG start codon, in the middle of the first exon of the AtTRE1 gene (FIG. 1, Panel A). T3 descendants showed 3:1 (resistant-susceptible) segregation on KAN selection media, consistent with a single insertion. Homozygous lines were obtained and confirmed by PCR (Ds3, AtTRELP2-RP2 primer set) and the CSHL predicted insertion site was confirmed. Semi-quantitative RT-PCR (primer set AtTREP1fw-rv) and real-time PCR (primer set AtTREP2fw-rv, FIG. 1, Panel B) revealed strong down-regulation of AtTRE1 in the homozygous GT_{—}16843 plants, hereinafter designed as “GT tre KO” plants.

The SALK_{—}151791 line (A. thaliana, ecotype Col) (FIG. 1, Panel A), originated from the SALK Institute (Alonso et al., 2003), was available from the Arabidopsis Biological Resource Center (ABRC), and ordered via NASC. From the ordered seeds, we selected plants, homozygous for a single T-DNA insertion in the AtTRE1 gDNA. After KAN selection, plants were checked for homozygousity with primers Lbal and AtTRELP1-RP1 primer set (designed with SIGnAL). Flanking T-DNA insertion sequences were obtained 304 bp upstream of the start codon (TAIR predicted 211 bp in front of the ATG start codon). Surprisingly, semi-quantitative RT-PCR (AtTREP1fw-rv) revealed higher AtTRE1 expression in the mutants, suggesting OX instead of KO. This was confirmed by real time-PCR (FIG. 1, Panel C, homozygous SALK_{—}151791 plants are hereinafter designed as SA TRE OX). Closer inspection of the left border of the T-DNA reveals a 35S CaMV promoter, which might be responsible for this overexpression. Also, insertion of the T-DNA in an important region for transcription regulation (e.g., a repressor binding site) is possible.

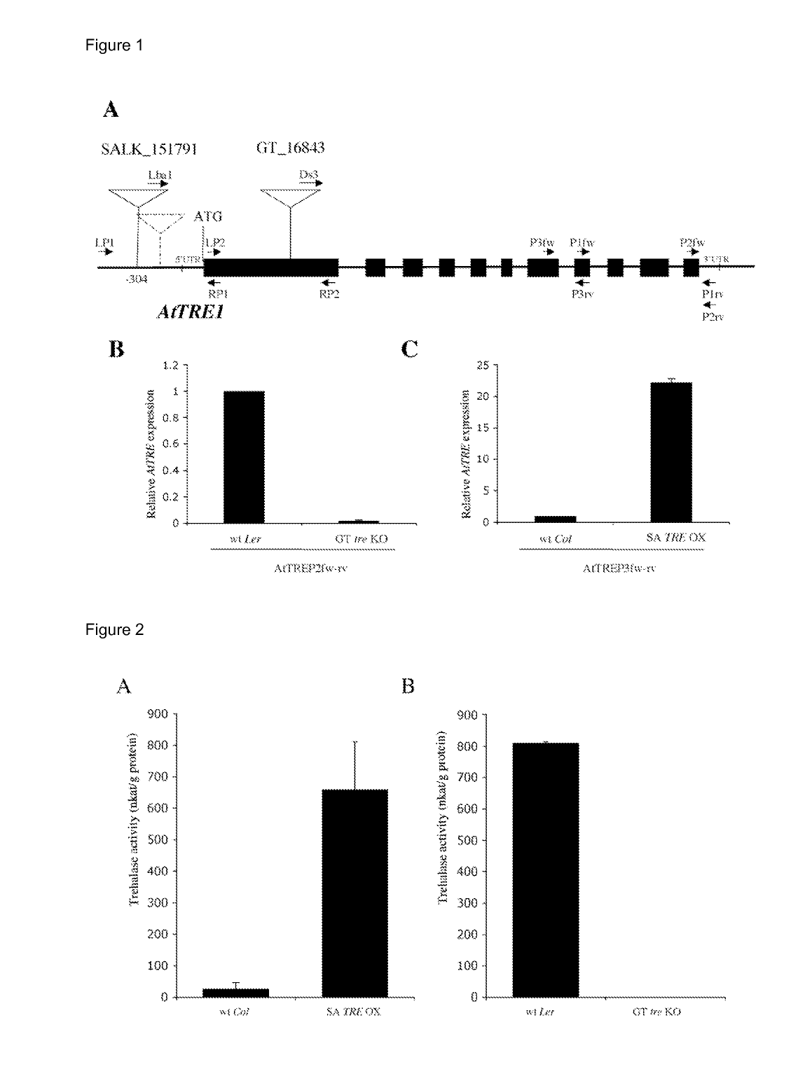

Measurements of trehalase activity in different plant tissues demonstrated that SA TRE OX plants exhibited much higher trehalase activity than control wild-type plants. For example, up to 25 times more trehalase activity was observed in adult leaves of TRE OX, compared to wild-type Col leaves (FIG. 2, Panel A). In contrast, very low to no detectable trehalase activity was measured in the leaves and siliques (FIG. 2, Panel B) of the GT tre KO plants compared to the control plants.

### Example 2

### Phenotypic Characterization of AtTRE1 Mutants

Trehalose causes dramatic effects on plant metabolism, growth and development. Validamycin A (Val A) specifically inhibits trehalase activity and prohibits the use of trehalose as an extra source of glucose. It is worth noting that, while Val A treatment alters adult plant stress and reproduction responses and even causes phytotoxicity, no phenotypic irregularities are observed in seedlings (Wingler et al., 2000; Müller et al., 2001; Ishikawa et al., 2005; 2007; Ramon et al., 2007).

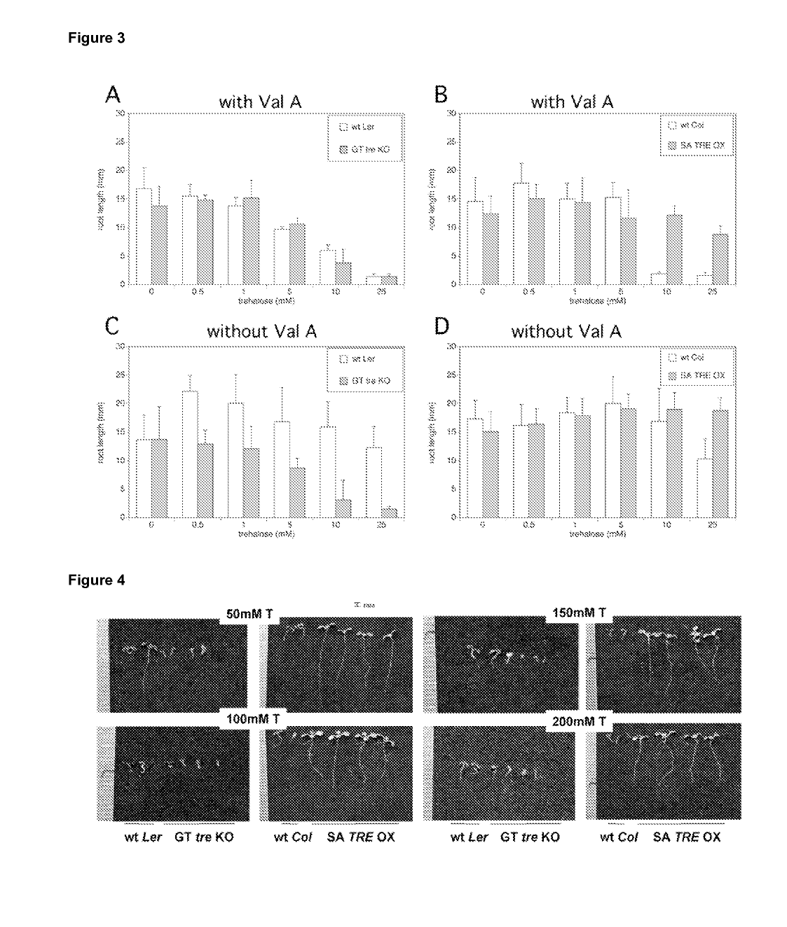

In our growth assay, no extra sugar was added to the media. As known from literature, gradually increased levels of trehalose in the presence of Val A, cause increased inhibition of root growth and development of wild-type seedlings (FIG. 3, Panels A, B). The sensitivity to trehalose differed between the ecotypes Ler and Col, with a rather gradual inhibitory effect in Ler wild-type plants, and a more dramatic decrease in root length of Col wild-type plants at 10 mM trehalose. GT tre KO lines exhibited similar growth inhibition on trehalose as wild-type Ler seedlings in the presence of Val A (FIG. 3, Panel A), consistent with lack of trehalase activity in both plants. In contrast, SA TRE OX plants were significantly more resistant to higher trehalose levels than wild-type controls in the presence of Val A (FIG. 3, Panel B), suggesting that the amount of Val A (10 μM) is not sufficient to inhibit increased trehalase activity in the OX lines.

In the absence of Val A, external supplied trehalose can be used as an extra glucose source for growth. On high trehalose concentrations, wild-type seedlings arrest with development shortly after germination, show altered cotyledons extension, and develop only short primary roots (Wingler et al., 2000).

Interestingly, at low trehalose contents, GT tre KO seedlings do not seem to be able to use the extra glucose source for growth (FIG. 3, Panel C), because of total lack of trehalase activity. Moreover, with increasing trehalose concentrations, KO mutants are significantly more affected in development, compared to wild-type Ler controls (FIG. 3, Panel C, FIG. 4). In contrast, SA TRE OX plants seem to be insensitive to high trehalose concentrations compared to wild-type seedlings (FIG. 3, Panel D, FIG. 4).

Mutants in trehalose metabolism have been reported to exhibit sensitivity to supplemented sugars, and T6P has been highlighted as an important regulator for carbohydrate utilization (Schluepmann et al., 2003; Avonce et al., 2004).

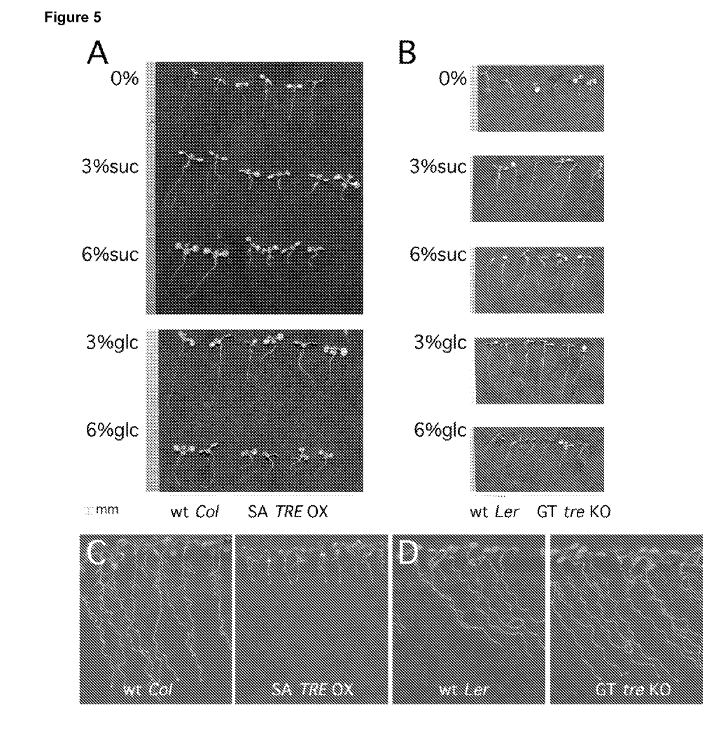

When grown on gradually increasing sugar concentrations, SA TRE OX seedlings exhibit similar leaf and hypocotyl development as control seedlings (horizontally orientated plates, 16 hours-8 hours day/night cycle (FIG. 5, Panel A). However, root growth of SA TRE OX seedlings was significantly affected, with reduced length and increased root branching. This aberrant phenotype was also observed on vertically oriented plates in continuous light with 1% sucrose (FIG. 5, Panel C). However, the mutants displayed a somewhat accelerated root growth on media without sugars. All together, it seems that overexpressing trehalase allows seedlings to efficiently use carbon supplies in low energy conditions, but makes them unable to deal with high energy conditions. In contrast, a slightly longer root growth of GT tre KO could be observed when more energy was available (FIG. 5, Panels B, D), and interestingly, GT tre KO plants seemed to germinate slower in media without sucrose.

Plants engineered in trehalose metabolism typically exhibit dramatic phenotypic irregularities, with altered physiology and morphology. In addition, Val A treatment can alter plant reproduction and can even cause phytotoxicity (Müller et al., 1995; Müller et al., 2001; Ishikawa et al., 2005; 2007).

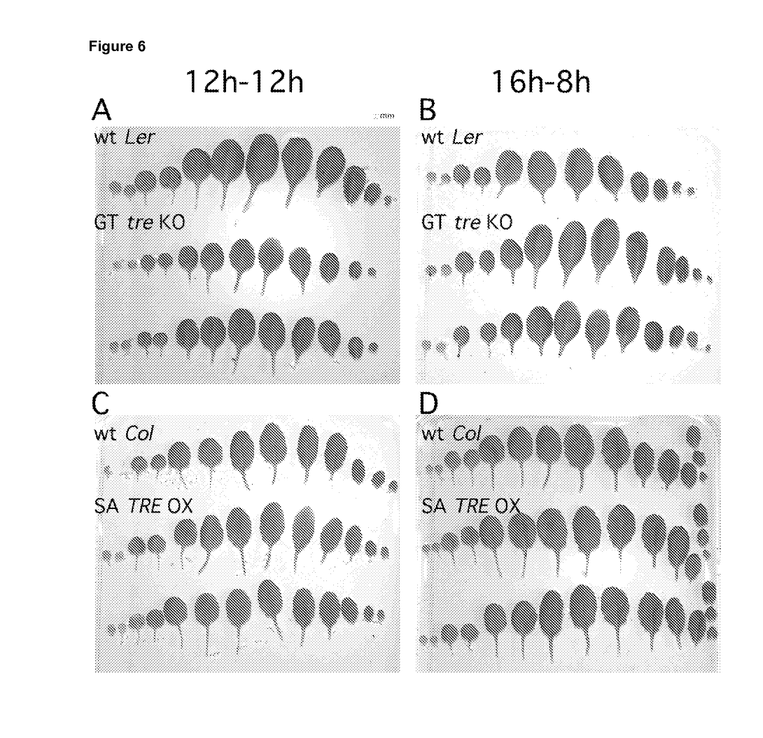

However, to our surprise, altering endogenous trehalase activities did not seem to cause severe phenotypic irregularities in Arabidopsis. We did notice a slower germination rate of GT tre KO seeds in soil in a 12 hours-12 hours day/night cycle, and this phenotype was abrogated when increasing the light period to 16 hours. Moreover, in a 12 hours-12 hours day/night cycle, leaves of the GT tre KO plants were smaller (FIG. 6, Panel A), and when increasing the day length, the growth rate of these KO plants increased (FIG. 6, Panel B, growth stages according to Boyes et al. (2001), 1.10 for wt and 1.11 for KO).

This observation again suggests that growth of plants, altered in trehalase activity, strongly depends on the available energy. GT tre KO plants seem to have problems to cope with low energy conditions, whereas higher growth rates are achieved when more energy is available, e.g., through longer photosynthesis or supplied sugars. In contrast, SA TRE OX plants grow slightly better than controls in low carbohydrate conditions (FIG. 6, Panel C, e.g., rosette leaf nr 9), while at a 16-hour-8-hour day/night cycle, no obvious growth phenotype was observed (FIG. 6, Panel D).

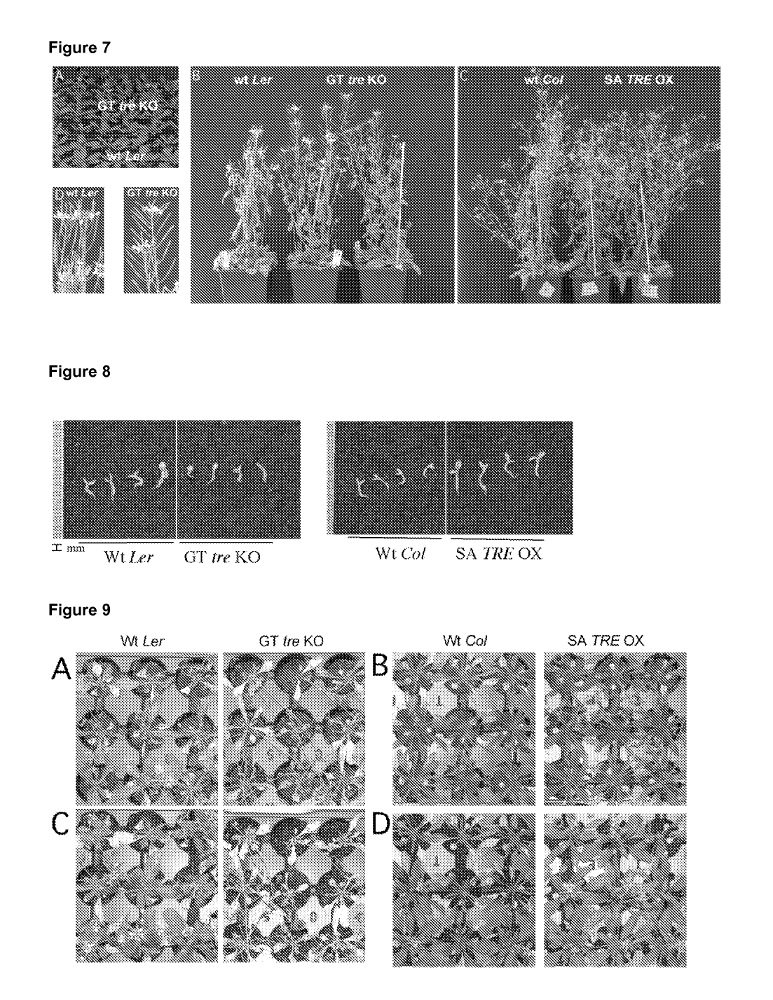

Interestingly, GT tre KO plants started flowering earlier than wild-types (16 hours-8 hours day/night cycle) (FIG. 7, Panel A), displayed longer inflorescence stems, and altered silique orientation (FIG. 7, Panels B and D). Stems of SA TRE OX plants (FIG. 7, Panel C) appeared somewhat smaller, but no obvious other phenotypes could be observed.

### Example 3

### Trehalase and Stress Responses

Given trehalose-modified plants performed better in multiple stress treatments, we tested trehalase OX and KO plants under variable stress conditions.

When performing heat-stress during germinating (33° C., continuous low light, 1× MS, 1% suc), we noticed a better early seedling development of SA TRE OX seeds, compared to wild-type seeds, in contrast to GT tre KO lines (FIG. 8).

These results, together with ample circumstantial evidence of pivotal roles of trehalose metabolism in abiotic stress responses, give us incentives to look closer to more environmental stresses such as drought and dehydration stresses in the vegetative stage. Four-week-old KO and OX plants grown in a 16-hour/8-hour day/night cycle (in soil with controlled watering regime), were subjected to drought stress for two weeks and then rewatered. In contrast to the performances of GT tre KO lines, the SA TRE OX plants withstand and recovered much better the dehydration stress (FIG. 9).

### Example 4

### AtTRE1 Gene Expression Analysis and Subcellular Localization

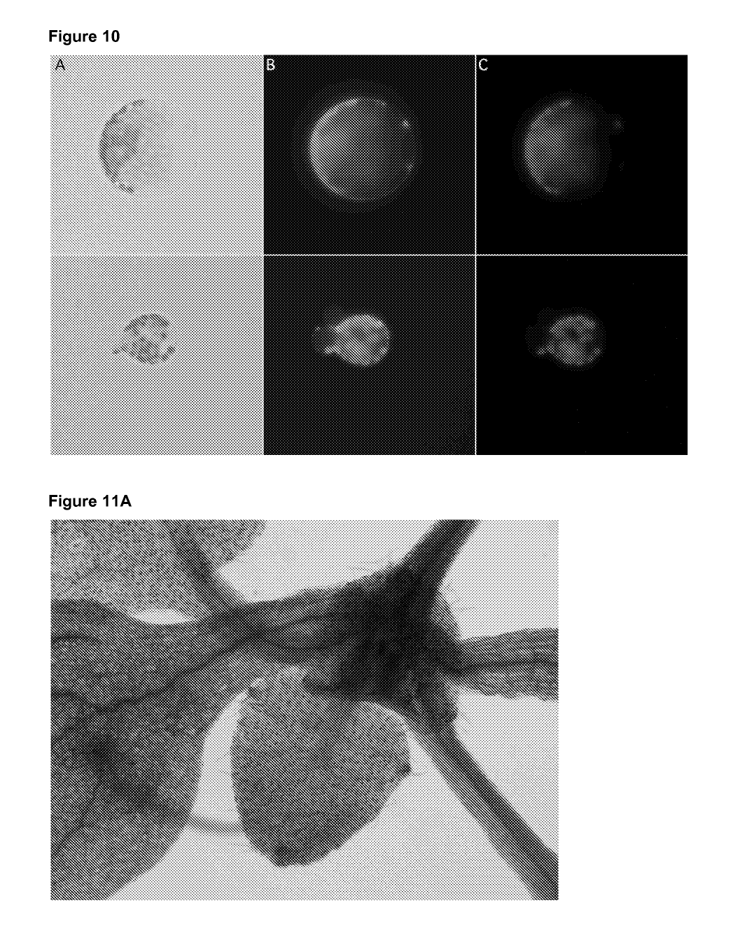

Given trehalase controls extracellular trehalose levels, but seems also to regulate plant trehalose (sugar) metabolism, we analyzed the fluorescence of AtTRE1-GFP constructs in Arabidopsis mesophyll protoplasts, to gain more insight in the exact location of trehalase. AtTRE1 seemed to be mainly localized in the plasma-membrane (FIG. 10). This result was recently confirmed and published by Frison and coworkers (2007).

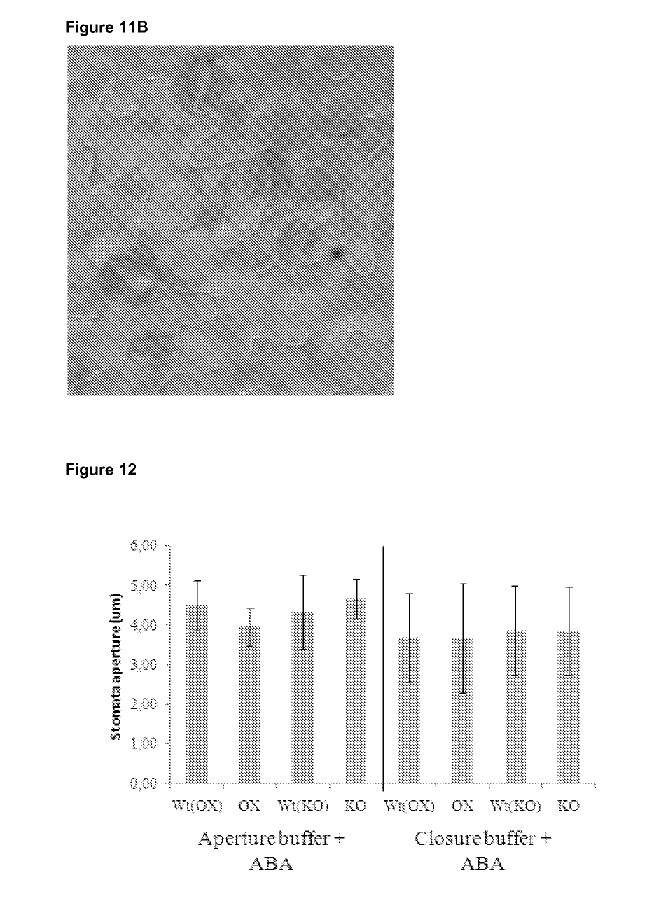

The expression was further analyzed by fusing the trehalase promoter to a GUS/GFP construct and analyzing the plants after GUS staining. In leafs and leaf primordia, a clear specificity of the expression is seen in the stomatal guard cells (FIGS. 11A and 11B); expression could also be detected in the flowers (in stomata in sepals of inflorescence, stomata in pedicel of inflorescence, petal veins and chalaza in siliques) and in the root tips.

### Example 5

### Trehalose Expression Controls Number and Opening of Stomata

To analyze further the effect of modulation of trehalase expression on stomata, mutants of AtTRE1 were grown in soil under normal moist conditions in a growth room. 21 days after sowing, Leaf 1 or Leaf 2 were cut out of the plant and submerged for the abaxial side in aperture buffer (10 mM MES, 10 mM CaCL2, 10 mM KCl, pH 6.1) under light conditions, or closure buffer (10 mM MES, 10 mM CaCL2, pH 6.1) under dark conditions. After two hours, leaves were submerged in aperture buffer containing 20 uM ABA under light conditions for 2 hours, the abaxial epidermis was mounted on a slide with double-sided tape and stomata aperture measurements (width) were recorded under a microscope, n=50. Trehalase overexpression plants showed a decrease in opening of stomata in aperture buffer, whereas in closure buffer, no significant difference between the wild-types, overexpression of KO plant could be detected (FIG. 12).

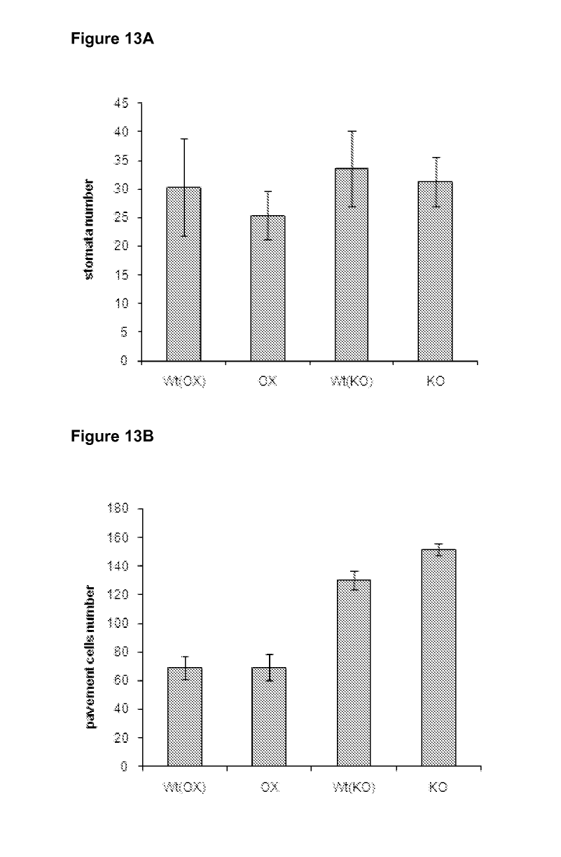

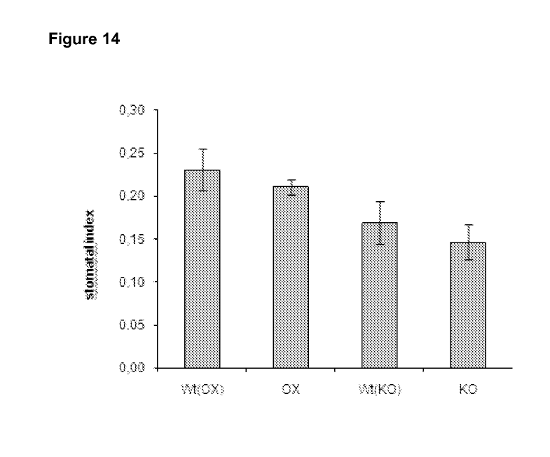

To analyze the effect of trehalase expression on number of stomata, mutants of AtTRE1 were grown on ½ MS, 1% sucrose horizontal plates, under 16 hours light/8 hours dark regime for 21 days. Leaf 1 or Leaf 2 were cut off and cleared in lactic acid for 2 days under constant shaking. Leaves were put on a slide and pictures (0.142 mm²) of 2 areas (tip and base) of each leaf were taken to count stomata and pavement cells, n=10 and to calculate the stomatal index. The stomatal index corresponds to the ratio of stomata per total number of cells in the epidermis (# stomata/(2×# stomata+# pavement cells). Compared with their respective wild-types, there is a slight decrease in the number of stomata (FIG. 13A) and in stomatal index (FIG. 14). Whereas, for the overexpressing plant no influence on the number of pavement cells was seen, the KO showed a small increase in pavement cell number (FIG. 13B). The results indicate that the trehalase modulates drought resistance by modulating the opening of the stomatal guard cells, and/or the number of stomatal guard cells per surface unit and/or the stomatal index.

### REFERENCES

Alonso J. M., A. N. Stepanova, T. J. Leisse, C. J. Kim, H. Chen, P. Shinn, D. K. Stevenson, J. Zimmerman, P. Barajas, R. Cheuk, C. Gadrinab, C. Heller, A. Jeske, E. Koesema, C. C. Meyers, H. Parker, L. Prednis, Y. Ansari, N. Choy, H. Deen, M. Geralt, N. Hazari, E. Horn, M. Karnes, C. Mulholland, R. Ndubaku, I. Schmidt, P. Guzman, L. Aguilar-Henonin, M. Schmid, D. Weigel, D. E. Carter, T. Marchand, E. Risseeuw, D. Brogden, A. Zeko, W. L. Crosby, C. C. Berry, and J. R. Ecker (2003). Genome-wide insertional mutagenesis of Arabidopsis thaliana. Science 301:653-657.
Avonce N., B. Leyman, J. O. Mascorro-Gallardo, P. Van Dijck, J. M. Thevelein, and G. Iturriaga (2004). The Arabidopsis trehalose-6-P synthase AtTPS1 gene is a regulator of glucose, abscisic acid, and stress signaling. Plant Physiol 136:3649-3659.
Boyes D. C., A. M. Zayed, R. Ascenzi, A. J. McCaskill, N. E. Hoffman, K. R. Davis, and J. Gorlach (2001). Growth stage-based phenotypic analysis of Arabidopsis: a model for high throughput functional genomics in plants. Plant Cell 13:1499-1510.
Del Sal G., G. Manfioletti, and C. Schneider (1988). The CTAB-DNA precipitation method: a common mini-scale preparation of template DNA from phagemids, phages or plasmids suitable for sequencing. Biotechniques 7:514-520.
Frison M., J. L. Parrou, D. Guillaumot, D. Masquelier, J. Francois, F. Chaumont, and H. Batoko (2007). The Arabidopsis thaliana trehalase is a plasma membrane-bound enzyme with extracellular activity. FEBS Lett 581:4010-4016.
Hauser M. T., A. Morikami, and P. N. Benfey (1995). Conditional root expansion mutants of Arabidopsis. Development 121:1237-1252.
Hwang I. and J. Sheen (2001). Two-component circuitry in Arabidopsis cytokinin signal transduction. Nature 413:383-389.
Ishikawa R., K. Shirouzu, H. Nakashita, T. Teraoka, and T. Arie (2007). Control efficacy of validamycin A against Fusarium wilt correlated with the severity of phytotoxic necrosis formed on tomato tissues. J. Pestic. Sci. 32.
Ishikawa R., K. Shirouzu, H. Nakashita, H. Y. Lee, T. Motoyama, I. Yamaguchi, T. Teraoka, and T. Arie (2005). Foliar Spray of Validamycin A or Validoxylamine A Controls Tomato Fusarium Wilt. Phytopathology 95:1209-1216.
Jang I. C., S. J. Oh, J. S. Seo, W. B. Choi, S. I. Song, C. H. Kim, Y. S. Kim, H. S. Seo, Y. D Choi, B. H. Nahm, and J. K. Kim (2003). Expression of bifunctional fusion of Escherichia coli gene for trehalose-6-phosphate synthase and trehalose-6-phosphate phosphatase in transgenic rice plants increases trehalose accumulation and abiotic stress tolerance without stunting growth. Plant Physiology 131:516-524.
Kovtun Y., W. L. Chiu, G. Tena, and J. Sheen (2000). Functional analysis of oxidative stress-activated mitogen-activated protein kinase cascade in plants. Proc. Natl. Acad. Sci. U.S.A. 97:2940-2945.
Kovtun Y., W. L. Chiu, W. Zeng, and J. Sheen (1998). Suppression of auxin signal transduction by a MAPK cascade in higher plants. Nature 395:716-720.
Liu Y. G., N. Mitsukawa, T. Oosumi, and R. F. Whittier (1995). Efficient isolation and mapping of Arabidopsis thaliana T-DNA insert junctions by thermal asymmetric interlaced PCR. Plant J. 8:457-463.
Lowry O. H., N. J. Rosebrough, A. L. Farr, and R. J. Randall (1951). Protein measurement with the Folin phenol reagent. J. Biol. Chem. 193:265-275.
Martienssen R. (1998). Functional genomics: probing plant gene function and expression with transposons. Proc. Natl. Acad. Sci. 95:2021-2026.
Müller J., R. A. Aeschbacher, A. Wingler, T. Boller, and A. Wiemken (2001a). Trehalose and trehalase in Arabidopsis. Plant Physiol. 125:1086-1093.
Müller J., T. Boller, and A. Wiemken (1995b). Effects of validamycin A, a potent trehalase inhibitor, and phytohormones on trehalose metabolism in roots and nodules of soybean and cowpea. Planta 197:362-368.
Ramon M., F. Rolland, J. M. Thevelein, P. Van Dijck, and B. Leyman (2007). ABI4 mediates the effects of exogenous trehalose on Arabidopsis growth and starch breakdown. Plant Mol. Biol. 63:195-206.
Schluepmann H., T. Pellny, A. van Dijken, S. Smeekens, and M. Paul (2003). Trehalose 6-phosphate is indispensable for carbohydrate utilization and growth in Arabidopsis thaliana. Proc. Natl. Acad. Sci. U.S.A. 100:6849-6854.
Suarez R., A. Wong, M. Ramirez, A. Barraza, C. Orozco Mdel, M. A. Cevallos, M. Lara, G. Hernandez, and G. Iturriaga (2008). Improvement of drought resistance and grain yield in common bean by overexpressing trehalose-6-phosphate synthase in rhizobia. Mol. Plant Microbe Interact. 21:958-966.
Sundaresan V., P. Springer, T. Volpe, S. Haward, J. D. Jones, C. Dean, H. Ma, and R. Martienssen (1995). Patterns of gene action in plant development revealed by enhancer trap and gene trap transposable elements. Genes Dev. 9:1797-1810.
Wingler A., T. Fritzius, A. Wiemken, T. Boller, and R. A. Aeschbacher (2000). Trehalose induces the ADP-glucose pyrophosphorylase gene, ApL3, and starch synthesis in Arabidopsis. Plant Physiol. 124:105-114.
Ye G. N., D. Stone, S. Z. Pang, W. Creely, K. Gonzalez, and M. Hinchee (1999). Arabidopsis ovule is the target for Agrobacterium in planta vacuum infiltration transformation. Plant J. 19:249-257.
Yoo S. D., Y. Cho, and J. Sheen (2007). Arabidopsis mesophyll protoplasts: a versatile cell system for transient gene expression analysis. Nature Protocols 2:1565-1572.

1. (canceled)

2. A method of modulating drought tolerance and/or salt tolerance in a plant comprising a recombinant trehalase gene, the method comprising:
overexpression of said recombinant trehalase gene so as to provide drought tolerance and/or salt tolerance in the plant.

3. The method according to claim 2, wherein modulation comprises obtaining drought tolerance and/or salt tolerance in the plant.

4. The method according to claim 2 to obtain improved growth of the plant upon rewatering of the plant after a drought period.

5. The method according to claim 3, wherein said overexpression is specific for stomatal guard cells of the plant.

6. A method of modulating stomatal guard cell function, the method comprising:
utilizing a recombinant trehalase gene to modulate the function of the stomatal guard cell.

7. The method according to claim 6, wherein said recombinant trehalase gene is overexpressed in the stomatal guard cell.

8. The method according to claim 7, wherein said modulation of the stomatal function comprises a limitation of the opening of the stomata.

9. The method according to claim 4, wherein the overexpression is specific for a stomatal guard cell.
